(19) United States
(12) Patent Application Publication
(10) Pub. No.: US 2026/0223875 A1
(43) Pub. Date: Aug. 6, 2026

(54) USE OF FORMULATIONS OF CURCUMINOIDS AS AN ADDITIVE TO A FERMENTED DAIRY PRODUCT, METHOD OF EXTENDING THE SHELF LIFE OF THE FERMENTED DAIRY PRODUCT AND THE PRODUCT OBTAINED BY THIS METHOD

(71) Applicant: DAIRY BIOTECHNOLOGIES SP. z o.o., Pulawy (PL)

(72) Inventors: Jakub URBANSKI, Warszawa (PL); Magdalena BUNIOWSKA-OLEJNIK, Stary Dzikow (PL); Maciej BANACH, Wola Grzymkowa (PL); Pawel BIEGANOWSKI, Warszawa (PL); Marcin JASKULA, Szamotuly (PL)

(21) Appl. No.: 19/155,075

(22) PCT Filed: Sep. 4, 2023
(86) PCT No.: PCT/PL2023/050072

§ 371 Date: Aug. 8, 2025

(30) Foreign Application Priority Data
Feb. 8, 2023 (PL) P.443720

(51) Int. Cl. A23C 9/13; A23B 2/733; A23C 9/137

(52) U.S. Cl. CPC A23C 9/1307; A23B 2/733; A23C 9/137

(57) ABSTRACT

The object of the invention is the use of formulations of curcuminoids as a technological additive extending the shelf life of fermented dairy products, as agent with antimicrobial activity against pathogenic microorganisms and, at the same time, stimulating the growth of probiotic microorganisms in fermented dairy products, as an agent improving the organoleptic and taste properties of a fermented dairy product, a method of extending the shelf life of a fermented dairy product and a fermented dairy product with extended shelf life obtained by this method.

The invention enables production of probiotic-nutraceutical dairy products with extended shelf life and edibility, comprising live probiotic strains of microorganisms and curcuminoids.

### CROSS REFERENCE TO RELATED APPLICATIONS

This U.S. Non-Provisional patent application is a national stage application of the International Application PCT/PL2023/050072, filed on Sep. 4, 2023, which in turn claims priority to Polish Patent Application PL443720, filed on Feb. 8, 2023 at the Polish Patent Office, and the disclosure of which is incorporated herein by reference.

### DESCRIPTION

### Technical Field

An object of the invention is a use of a water dispersed formulation of curcuminoids as a technological additive extending the shelf life (prolonging durability) of fermented dairy products, as an agent having antimicrobial activity against pathogenic microorganisms and, at the same time, stimulating the growth of probiotic microorganisms in fermented dairy products, as an agent improving organoleptic and taste properties of a fermented dairy product, a method for extending the shelf life of the fermented dairy product, including the fermented dairy product with extended shelf life obtained by this method.

The invention enables the production of nutraceutical dairy products having extended shelf life and edibility, containing live probiotic strains of microorganisms, by using a formulation of a highly purified turmeric extract, odor-free and free of the spicy-burning taste typical for turmeric extracts, dispersing in water to produce dairy food products, including functional products having extended shelf life and edibility.

### State of the Art

One of the most visible trends in food production and new food products in recent years is the introduction of functional food products to the market. Functional food is food of natural origin that can be enriched or modified. In addition to providing nutrients, it has a positive effect on health. It may reduce the risk of developing certain diseases, especially the so-called lifestyle diseases, or generally improve health and well-being. Its main characteristic is that it must resemble conventional food. Furthermore, it must have beneficial effects in amounts that can be supplied by a normal, well-balanced diet. Functional food cannot be either capsules or tablets. In functional foods, carriers of bioactive substances are regularly consumed products: dairy products, cereal products and fruit juices. In 1996, the European Commission initiated the project-Functional Food Science in Europe (FUFOSE), which aim was to develop a scientific basis for the concept of functional food. In the summary of the project, it was assumed that food can be called functional if, in addition to its usual nutritional effect, it has a proven beneficial effect on one or more body functions.

Due to their widespread availability, relatively low price and documented nutritional properties, fermented dairy products have a great potential as functional foods. Probiotic products are, by definition, functional foods due to the content of probiotic strains. Probiotic products account for approximately 15% of the fermented dairy products market.

Probiotic yogurts are potentially the basis for more advanced functional products—in the form of probiotic-nutraceutical dairy products. However, the development of this type of products requires overcoming limitations resulting from the state of the art and potential interferences between probiotics and nutraceutical additives. This applies to both the negative impact of nutraceuticals on the stability of probiotic bacteria, potential contamination of probiotic yogurts with nutraceutical additives (especially in the case of nutraceuticals of plant origin), the impact of nutraceuticals on the sensory and organoleptic parameters of the final product, and the negative impact of active probiotic bacteria on the nutraceutical.

An undoubted challenge is to maintain the characteristics of the final product, such as acceptable shelf-life and microbiological stability. Fermented milk drinks are milk products with a liquid or semi-liquid consistency, obtained by fermenting a milk mixture using special microorganisms included in starter preparations. Fermented milk drinks are divided into three types depending on their shelf life: fresh fermented milk drinks with a short shelf life, those with an extended shelf life and heat-treated fermented milk drinks. At the same time, because fermented dairy products spoil due to the threat of fungi and yeast, their long-term shelf life is an issue. The most popular way to increase the stability of fermented milk products is the thermalization process of the mixture, which involves processing at 60-70° C. with a holding period of at least 30 min. However, this method reduces the sensory properties of the product during storage and may contribute to a partial denaturation of milk proteins. Other known methods include the selection of special types of packaging, the use of high pressure treatment, excessive irradiation, high temperature treatment, thermosonication treatment and ozone treatment. The main problem of such methods is their negative impact on the microorganisms in the fermentation preparations, in particular on their viability (Dash el al. 2022; Kennang et al. 2020).

For the mass production of fermented dairy products, it is also common to use preservatives that inhibit the decomposition processes occurring in non-living cells and limit the activity of metabolic processes in living cells, thereby ensuring long-term storage of the product. In the case of selecting preservatives, manufacturers should consider the mechanism of action because each preservative acts selectively on a specific type of microorganism. In the absence of a scientific explanation for the selection of preservatives, they significantly inhibit the activity of the lactic acid microflora of fermented milk drinks, even in minimal doses (Var et al., 2004). In the dairy industry, the most known preservatives are potassium sorbate, benzoic acid and sorbic acid.

Natural preservatives are lactic acid microorganisms that produce antimicrobial metabolites during milk fermentation, in particular organic acids such as propionic, acetic and lactic acids (Zapaśnik et al., 2022). They create an unfavorable environment for the development of pathogenic microorganisms (Kukhtyn et al., 2018). Lactic acid bacteria are a diverse group of beneficial bacteria that have been unknowingly used by humans for thousands of years as starter cultures for the preparation of various fermented dairy products, including yogurt, kefir, sour cream, ryazhenka, acidophilous milk, curdled milk, ayran, kumis (koumiss) and other (Petrova et al., 2021).

The growing interest of consumers in natural products that do not contain artificially synthesized additives and preservatives has led to renewed interest in the use of natural antimicrobials in food products, in particular fermented milk drinks (Batiha et al., 2021; Mykhalevych et al., 2022). It is therefore advisable to look for natural ingredients which, due to their properties, including in combination with lactic acid microorganisms, can inhibit the development of pathogenic microorganisms and extend the shelf life of fermented milk drinks.

Undoubtedly, an interesting and very promising candidate for the production of probiotic and nutraceutical dairy products is curcumin, a polyphenol known and used for thousands of years in the medical traditions of the Far East, naturally contained in turmeric rhizomes. Curcuma longa, commonly called turmeric, is a plant of the Zingiberaceae family originating in southern Asia. Dried turmeric rhizomes contain 3-5% of essential oils (ethereal oils) and 0.02-6.0% of curcuminoids, such as curcumin, demethoxy-curcumin and bisdemethoxy-curcumin. These curcuminoids may occur in different amounts and proportions to each other, which depends on biological and environmental factors related to cultivation, and in the case of oleoresin or extracts, on the technology of their production. In addition to its coloring and spice properties, turmeric, and especially the curcuminoids contained within it, has various medicinal properties confirmed by pharmacological tests. Turmeric extracts have anti-inflammatory, antifungal, antiviral (Jennings and Parks, 2020) and anticancer properties (Amalraj et al., 2017; Yamamoto et al., 1997; Apisariyakul et al., 1995; Ruby et al., 1995). In order for curcuminoids to affect living organisms (humans and animals), they must first be delivered to the body and then absorbed from the digestive tract into the bloodstream (Amalraj et al., 2017; Jäger et al., 2014).

Due to their potential health-promoting properties, curcuminoids constitute an attractive, both from a commercial and functional point of view, food additive to i.a. dairy products, however, due to the very low solubility in water and poor dispersibility, and in the case of dried turmeric, also the low percentage of curcuminoids, their use in the form of turmeric or a standardized curcuminoid extract is very limited.

Curcuminoids are compounds that do not dissolve in water and, moreover, their solubility in plant and animal fats is very limited and does not exceed 1 mg ml-1, which, on the one hand, significantly hinders the bioavailability of curcuminoids from the digestive tract, and on the other hand, makes it difficult to use them in technological processes related to dairy production.

It was shown that the dispersion of curcuminoids in an aqueous environment significantly improves their bioavailability (Jager et al., 2014). Additionally, the aim of developing formulations of curcuminoids soluble or dispersible in water is to facilitate their combination with an appropriate flavor and fragrance composition, thanks to which they can be much more willingly consumed by humans and animals. Curcuminoids in concentrated form, like powdered turmeric itself, have a very intense, characteristic taste and are bitter, which practically disqualifies the possibility of consuming them directly. In this form, they can be either encapsulated or used as a food additive.

Many methods of obtaining water dispersible formulations of curcuminoids are described in the literature. A very common way to obtain water dispersible curcuminoids is to dissolve them in 70-80% ethanol together with β-cyclodextrin, followed by long heating and mixing for at least 4 h at 70° C. (Mangolim et al., 2014; Marcolino et al., 2011). Solvents (ethanol and water) are most often removed by spray drying, but also by vacuum evaporation or freeze-drying. This method of obtaining water dispersible curcuminoids has disadvantages, as it causes unfavorable chemical transformations of curcuminoids in a water-ethanol environment related to their thermal decomposition. Research by Wang et al. (1999) showed that curcuminoids decompose in an aquatic environment under the influence of increased temperature, even at the most favorable pH of the environment (curcuminoids show the greatest stability at pH between 3 and 6). Wang et al., (1999) showed that heating curcumin at 37° C. for 2 h causes as much as 10% loss of this compound. Creating formulations of water dispersible curcuminoids should be carried out at as low temperatures as possible, especially in the process of combining them with the water phase. An additional disadvantage of the formulation using β-cyclodextrin is its limited use in food production. β-cyclodextrin (E 459) can be used to produce e.g. beverages, but its content cannot exceed 1 g kg⁻¹ (EU Commission Regulation No. 1129/2011). This limits the possibility of introducing any amount of curcuminoids into the final product.

There are a number of formulations of curcuminoids available on the market with improved functional properties. CurQfen® produced by Akay Flavors & Aromatics Pvt. Ltd., MicroActive Curcumin produced by BioActives LLC (USA) contains approx. 25% of curcuminoids suspended in short-chain triacylglycerols with the addition of free fatty acids and polyglycerol esters. The formulation is stabilized with hydroxypropylmethylcellulose, sodium alginate and microcrystalline cellulose (Madhavi et al., 2014). This formulation was developed mainly for the production of tablets/capsules containing highly bioavailable curcumin. NovaSOL® produced by FRUTAROM from Israel is micellar curcumin with a concentration of approx. 7% in Tween-80 (Schiborr et al., 2014). The preparation cannot be used in food because Tween-80 is not approved for use in food in many countries, including the EU. CURCUGREEN® (BCM-95®) produced by Arjuna Natural Extracts Ltd., (India) is an essential oil consisting of 45% ar-turmerone and curcuminoids (Antony et al., 2008). CURCUGREEN® does not dissolve in water and is not suitable for the production of beverages. Curcumin C3 Complex®+Bioperine® produced by Sabinsa USA is a mixture of piperine and curcuminoids without a special formulation (Shoba et al., 1998). The preparation is not suitable for dispersion in water. CAVACURMIN® produced by Wacker Chemie AG, (Germany) contains approx. 15% of curcuminoids combined with γ-cyclodextrin (Purpura et al., 1998). This product disperses in water. Additionally, γ-cyclodextrin is restricted for use in food in the EU. Theracurmin™ produced by Theravalues Corp. (Japan) is a nano-colloidal preparation containing up to 30% curcuminoids. The preparation additionally contains glycerin and Ghatti gum (Sasaki et al., 2011). The product disperses well in water, is suitable for the production of beverages, however, at the moment, it cannot be used for food production because Ghatti gum is limited in food production as an additional substance in the EU. The NOMICUR L-100 preparation (NomiBiotech Corporation) is characterized by high dispersion, contains 25-30% of curcuminoids and is based on the use of stabilizers in the form of substances such as maltodextrin, saccharoglycerides and soy lecithin approved for use in the food industry. Similar features are characterized by NomiCUR beta, a highly dispersive curcumin formulation patented by Nomi Biotech Corporation containing turmeric extract, which contains at least 90% curcuminoids and is based on the use of stabilizers such as starch octenylsuccinate, hydroxypropylmethylcellulose and/or hydeoxypropylcellulose, β-glucan, a cellulose derivative with a viscosity above 5600 mPas (Patent PL241417).

All the commercially available curcumin formulations described above were designed to maximize the bioavailability of curcuminoids, only in some formulations, obtaining increased bioavailability was combined with increased dispersibility, which consequently allows the formulation to be used for the production of beverages.

### DISCLOSURE OF THE INVENTION

### Technical Problem

A technological problem arising from the state of the art is the short shelf life and edibility of yogurts and fermented dairy products containing probiotics, usually not exceeding several to a dozen or so days, as well as the lack of a technological additive of a nutraceutical nature, containing curcuminoids, which would allow obtaining a probiotic-nutraceutical yogurt with qualitative characteristics analogous or improved in relation to probiotic yogurts.

After the addition of the formulation of curcuminoids to the dairy product at both the pre-fermentation and post-fermentation stages, the product should be uniformly colored. Advantageously, the use of a formulation of curcuminoids should not negatively affect the organoleptic parameters of the product, such as taste and smell (scent), preferably all substances contained in the formulation should be allowed for use in food products, including in the EU, in accordance with the currently applicable regulations in this regard.

The use of formulations of curcuminoids as a technological additive preferably should not have an effect on the length and efficiency of the fermentation process, as well as the stability and reduction of the number of probiotic microorganisms.

### Aim of the Invention

The aim of the invention is to overcome the indicated inconveniences resulting from the state of the art. This aim was achieved by the unexpected development of a technological solution in the form of a technological additive for the production of fermented dairy products, in the form of a nutraceutical, which, within the range of curcuminoid concentrations permitted by EFSA (European Food Safety Authority) regulations, provides nutraceutical properties and, at the same time, ensures extension of the shelf life (durability) of dairy products and improvement of their organoleptic parameters.

The authors of the invention have unexpectedly found that the use of highly purified, water dispersible formulations of curcuminoids, containing min. 15% curcuminoids in dry matter, as a technological additive, stabilized with food additives approved for use in food products enables the production of probiotic yogurts, probiotic-nutraceutical yogurts with extended shelf life and edibility and improved organoleptic parameters in a standard technological process.

### Essence of the Invention

The invention relates to use of formulation of curcuminoids as a technological additive extending the shelf life of fermented dairy products, wherein the formulation of curcuminoids is a composition, which: in dry form comprises at least 20% w/w of curcuminoids, more preferably at least 25% w/w of curcuminoids; comprises no more than 0.01% w/w of essential oils; comprises a carrier of curcuminoids in the range of 60-70% w/w of the formulation, wherein the carrier of curcuminoids are sugars, polysaccharides, including saccharide polymers derived from starch, cellulose or mixtures thereof, approved for use in food products; comprises up to 20% w/w of an additional non-sugar excipient in a form approved for use in food products; the formulation disperses in water in at least 98%, preferably it shows 100% dispersion, wherein in the accelerated sedimentation assay, during centrifugation at a relative centrifugal force of 425 RCF for 10 min, no less than 80% of the formulation remains in the solution; the particle size of the dispersion for at least 95% of the particles is 1-2 μm; the formulation of curcuminoids is neutral in taste.

In the preferred use, the carrier of curcuminoids is selected from maltose, starch derivatives, starch octenylsuccinate, starch octenylsuccinate salt, monostarch phosphate, acetylated starch, acetylated distarch adipate, maltodextrin, cellulose derivatives, carboxymethylcellulose, hydroxypropylmethylcellulose and mixtures thereof.

In the preferred use, the additional non-sugar excipients are selected from citric acid, saccharoglycerides, beta glucan, lecithin and mixtures thereof.

In the preferred use, additional non-sugar excipients are up to 10% w/w of the formulation.

In the preferred use, the formulation of curcuminoids constituting a technological additive, when in dry form, is in the form of a powder, granules or a mixture thereof.

In the preferred use, the fermented dairy product is selected from yogurt, kefir, buttermilk, fermented milk, sour cream, ryazhenka, curdled milk, ayran, kumis, acidophilous milk.

In the preferred use, the formulation of curcuminoids as a technological additive that extends the shelf life of fermented dairy products is used in an amount of 0.1-0.25% w/w of curcuminoids in the fermented dairy product.

In the preferred use, the fermented dairy product is a probiotic-nutraceutical product comprising live cultures of probiotic strains in an amount of at least 10⁶ CFU/g, and in a 150 g portion of the fermented dairy product, it contains a dose of a nutraceutical in the form of curcuminoids, providing a person consuming it the amount of 2-4 mg/kg body weight, preferably the amount of 3±10% mg/kg body weight.

In the preferred use, the formulation of curcuminoids is a formulation in which the carrier of curcuminoids is a mixture of maltose, starch octenylsuccinate, maltodextrin, and the additional excipient is citric acid.

In the preferred use, the formulation of curcuminoids is a formulation, in which the carrier of curcuminoids is maltodextrin and the additional excipient is a mixture of saccharoglycerides and lecithin.

In the preferred use, the formulation of curcuminoids is a formulation in which the carrier of curcuminoids is a mixture of carboxymethylcellulose, starch octenylsuccinate, hydroxypropylmethylcellulose, and the additional excipient is beta glucan.

In the preferred use, the formulation of curcuminoids is formed in a process in which: dried Curcuma longa rhizome is subjected extraction for curcuminoids using an organic solvent, obtaining a highly purified curcuminoid extract with a content of min. 95% of curcuminoids, which extract is then dissolved in an organic solvent; homogenization of the curcuminoids solution with a hydrated bed of the carrier of curcuminoids consisting of sugars, polysaccharides including saccharide polymers derived from starch, cellulose or mixtures thereof, approved for use in food products is carried out, preferably supplementing the solution with additional non-sugar excipients; the obtained homogenate solution is dried, preferably by spray drying.

The invention also relates to a method for extending the shelf life of a fermented dairy product, comprising a step in which a) before the fermentation process of the dairy product, or b) after the fermentation process of a dairy product, a water dispersible formulation of curcuminoids is added as a technological additive extending the shelf life of fermented dairy products, in an amount ensuring 0.1-0.25% w/w of curcuminoids in the fermented dairy product; preferably, the amount of the additive does not exceed 1% w/w of the final dairy product, and the technological additive of the formulation of curcuminoids that extends the stability of fermented dairy products is the formulation of curcuminoids, which: in dry form it comprises at least 20% w/w, more preferably at least 25% w/w of curcuminoids; comprises no more than 0.01% w/w of essential oils; comprises a carrier of curcuminoids in the range of 60-70% w/w, wherein the carrier of curcuminoids are sugars, polysaccharides, including saccharide polymers derived from starch, cellulose or mixtures thereof, approved for use in food products; comprises up to 20% w/w, more preferably up to 10% w/w of an additional non-sugar excipient in a form approved for use in food products; disperses in water in at least 98%, preferably shows 100% dispersion, while in the accelerated sedimentation assay, during centrifugation at a relative centrifugal force of 425 RCF for 10 min, no less than 80% of the formulation remains in the solution; the particle size of the dispersion for at least 95% of the particles is 1-2 μm; the formulation of curcuminoids is neutral in taste.

In the preferred method for extending the shelf life of a fermented dairy product, the carrier of curcuminoids is selected from maltose, starch derivatives, starch octenylsuccinate, starch octenylsuccinate salt, monostarch phosphate, acetylated starch, acetylated distarch adipate, maltodextrin, cellulose derivatives, carboxymethylcellulose, hydroxypropylmethylcellulose and mixtures thereof.

In the preferred method for extending the shelf life of a fermented dairy product, the additional non-sugar excipients are selected from citric acid, saccharoglycerides, beta glucan, lecithin and mixtures thereof.

In the preferred method of extending the shelf life of a fermented dairy product, the formulation of curcuminoids constituting a technological additive, when in dry form, is in the form of powder, granules or a mixture thereof.

In the preferred method for extending the shelf life of a fermented dairy product, the fermented dairy product is selected from yogurt, kefir, buttermilk, fermented milk, sour cream, ryazhenka, curdled milk, ajran, kumis, acidophilous milk.

In the preferred method of extending the shelf life of a fermented dairy product, the fermented dairy product is a probiotic-nutraceutical product containing live cultures of probiotic strains in an amount of at least 10⁶ CFU/g, and in a 150 g portion of the fermented dairy product, it contains a dose of a nutraceutical in the form of curcuminoids, providing the person consuming it the amount of 2-4 mg/kg body weight, preferably the amount of 3±10% mg/kg body weight.

The invention also relates to a fermented dairy product with extended shelf life prepared by the method of the invention.

The fermented dairy product is preferably selected from yogurt, kefir, buttermilk, fermented milk, sour cream, ryazhenka, sour milk, ayran, kumis, acidophilous milk.

The fermented dairy product is preferably a probiotic-nutraceutical product containing live cultures of probiotic strains in an amount of at least 10⁶ CFU/g, and in a 150 g portion of the fermented dairy product, it contains a dose of a nutraceutical in the form of curcuminoids, providing the person consuming it the amount of 2-4 mg/kg body weight, preferably the amount of 3±10% mg/kg body weight.

The invention also relates to the use of a formulation of curcuminoids as an agent improving organoleptic and taste properties of a fermented dairy product, wherein the formulation of curcuminoids is a composition which: in dry form it comprises at least 20% w/w of curcuminoids, more preferably at least 25% w/w of curcuminoids; comprises no more than 0.01% w/w of essential oils; comprises a carrier of curcuminoids in the range of 60-70% w/w of the formulation, wherein the carrier of curcuminoids are sugars, polysaccharides, including saccharide polymers derived from starch, cellulose or mixtures thereof, approved for use in food products; comprises up to 20% w/w of an additional non-sugar excipient in a form approved for use in food products, the formulation disperses in water in at least 98%, preferably shows 100% dispersion, wherein in the accelerated sedimentation assay, during centrifugation at a relative centrifugal force of 425 RCF for 10 min, no less than 80% of the formulation remains in the solution; the particle size of the dispersion for at least 95% of the particles is 1-2 μm, the formulation of curcuminoids is neutral in taste; wherein the agent improving organoleptic and taste properties of the fermented dairy product is used as an agent imitating the taste of milk fat and/or as an agent enhancing the sweet taste sensation.

In the preferred use of formulations of curcuminoids as agent improving organoleptic and taste properties, the carrier of curcuminoids is selected from maltose, starch derivatives, starch octenylsuccinate, starch octenylsuccinate salt, monostarch phosphate, acetylated starch, acetylated distarch adipate, maltodextrin, cellulose derivatives, carboxymethylcellulose, hydroxypropylmethylcellulose and mixtures thereof.

In the preferred use of formulations of curcuminoids as an agent improving organoleptic and taste properties, additional non-sugar excipients are selected from citric acid, saccharoglycerides, beta glucan, lecithin and mixtures thereof.

The invention also relates to the use of formulations of curcuminoids as an agent with antimicrobial activity against pathogenic microorganisms and at the same time stimulating the growth of probiotic microorganisms in fermented dairy products; wherein the formulation of curcuminoids is a composition that: in dry form comprises at least 20% w/w, more preferably at least 25% w/w of curcuminoids; comprises no more than 0.01% w/w of essential oils; comprises a carrier of curcuminoids in the range of 60-70% w/w of the formulation, wherein the carrier of curcuminoids are sugars, polysaccharides, including saccharide polymers derived from starch, cellulose or mixtures thereof, approved for use in food products; comprises up to 20% w/w of an additional non-sugar excipient in a form approved for use in food products, the formulation disperses in water in at least 98%, preferably shows 100% dispersion, wherein in the accelerated sedimentation assay, during centrifugation at a relative centrifugal force of 425 RCF for 10 min, no less than 80% of the formulation remains in the solution; the particle size of the dispersion for at least 95% of the particles is 1-2 μm; the formulation of curcuminoids is neutral in taste.

In the preferred use of formulations of curcuminoids as an agent with antimicrobial activity against pathogenic microorganisms and, at the same time, stimulating the growth of probiotic microorganisms, the carrier of curcuminoids is selected from maltose, starch derivatives, starch octenylsuccinate, starch octenylsuccinate salt, monostarch phosphate, acetylated starch, acetylated distarch adipate, maltodextrin, cellulose derivatives, carboxymethylcellulose, hydroxypropylmethylcellulose and mixtures thereof.

In the preferred use of formulations of curcuminoids as an agent with antimicrobial activity against pathogenic microorganisms and at the same time stimulating the growth of probiotic microorganisms, additional non-sugar excipients are selected from citric acid, saccharoglycerides, beta glucan, lecithin and mixtures thereof.

In the preferred use of formulations of curcuminoids as an agent with antimicrobial activity against pathogenic microorganisms and at the same time stimulating the growth of probiotic microorganisms, the pathogenic microorganisms are selected from G+ bacteria: Staphylococcus aureus, Listeria monnocytogenes, Streptococcus pneumoniae; G− bacteria: Escherichia coli, Pseudomonas aeruginosa, Yersinia enterocolitica; yeasts: Candida albicans, Candida glabrata, Candida krusei, Candida tropicalis, fungi: Penicillium chrysogenum, Penicillium brevicompactum, Penicillium italicum, Penicillium aurantiogriseum; and the probiotic microorganisms are selected from Lactobacillus delbrueckii, Streptococcus thermophilus, Bifidobacterium animalis subsp. lactis, Lactobacillus acidophilus, Lactobacillus casei, Lactobacillus johnsonii.

The research conducted by the inventors has shown that the use of water dispersible formulations of highly purified curcuminoids as a technological additive in the probiotic dairy production process, comprising excipients approved for use in the production of food with GRAS (Generally Regarded as Safe) status, allows for obtaining products with new, non-obvious properties in the form of significantly extended shelf life and microbiological stability, as well as health-promoting properties resulting from the content of live probiotic strains and the nutraceutical-curcumin. The inventors found that the addition of a water dispersible formulation of highly purified curcuminoids, preferably a formulation with a neutral taste and smell (odour), and especially preferably comprising highly purified, standardized to min. 95% of the curcuminoid extract without essential oils, preferably containing min. 20% of curcuminoids, preferably comprising stabilizing additives, particularly preferably additives in the form of maltodextrin, saccharoglycerides and hydrolyzed soy lecithin, allows for microbiological stabilization of a probiotic yogurt, inhibiting the growth of fungi and molds and maintaining the count of probiotic bacteria. The obtained product shows stability for no less than 28 days.

The inventors conducted a preliminary research to define a set of desired physico-chemical features characterizing a potential technological additive in the form of a formulation of curcuminoids.

A total of 9 substances were analyzed, including commercially available formulations of curcuminoids such as: NovaSOL®, Cavacurmin, TheracurminCP, Curcugen, C3, Curcufen, NomiCUR L-100, NomiCUR beta formulation according to the PL241417 patent and the standardized curcuminoids extract.

Due to negative sensory features that may significantly affect the organoleptic and sensory parameters of the products, such as the intense taste and smell characteristic of the curcuminoid extract, the Curcugen, C3, Curcufen formulations were eliminated.

Then, due to the physical features, i.e. the lack of the ability to create stable dispersions, which constitutes an obstacle to the use of the formulation in technological processes related to the production of yogurt, it also turned out to have a negative impact on the organoleptic parameters and NovaSOL®, Cavacurmin, which hindered uniform dosing of the nutraceutical substance in the portioning process of the final product, were eliminated, in the case of NovaSOL®, the limitation was the relatively low content of curcuminoids, forcing the use of the additive in an amount exceeding 1% w/w of the final product.

TheracurminCP, NomiCUR L-100 formulations, NomiCU® beta formulations according to patent PL241417 were used in further studies.

The present invention, as illustrated below, may be suitably practiced in the absence of any of the elements or limitations, not specifically disclosed herein.

Whenever the term “comprising” or “containing” is used in this description and claims, it does not exclude other elements. For the purposes of the present invention, the term “consisting of” is considered a preferred embodiment of the term “comprising”, “containing”. If a group is defined below, which contains at least a number of embodiments, this should also be understood as disclosing a group, which preferably consists of only these embodiments.

The terms “about” or “approximately” in the context of the present invention stand for a range of accuracy that a person skilled in the art will understand to be sufficient to still provide the technical effect of the given feature. The term “typically” is to be understood as a deviation from the indicated numerical value of +10%, preferably +5%.

Technical terms are used according to their usual meaning. If a specific meaning is used, the meaning will be described in detail in relation to the context in which the term was used.

The terms “technological additive in the form of formulations of curcuminoids”, “technological additive of dispersible curcuminoids”, “technological additive of formulations of curcuminoids” are used interchangeably.

By the term “stability” is to be understood as the formation of a dispersed system with a dispersed phase particle size <1 μm. By stability is to be understood the formation of a dispersion system that is optically homogeneous, with no visible creaming or sedimentation process. The system is considered stable if during centrifuging in a centrifuge at 5000 rpm for 20 min and at the temperature of 25° C., the amount of sediment at the bottom of the tube does not exceed 30% relative to the initial weight of the water-dispersed powder formulation.

Dispersion of a technological additive in the form of a formulation of curcuminoids is to be understood as a system composed of at least two immiscible phases, of which at least one is a finely divided system (dispersed phase), dispersed in the second phase, with continuous character (dispersive phase), called the dispersion medium.

The solubility of a technological additive in the form of a formulation of curcuminoids is to be understood as the ability of a solid substance to dissolve in the liquid phase and produce a dispersion system forming a heterogeneous mixture.

The invention comprises use as a technological additive in the form of a formulation of curcuminoids for the production of dairy products to extend its shelf life, improving taste, to obtain nutraceuticals, especially fermented dairy products, all formulations constituting such an additive essentially having the features described below:
The formulation is stable under lactic fermentation conditions, including yogurt fermentation;
The formulation is in at least 98% dispersible in water, preferably shows 100% dispersion, assuming that in the accelerated sedimentation assay, during centrifugation at 425 RCF (relative centrifugal force), no less than 80% of the formulation remains in the solution for 10 min;
The formula comprises at least 20% w/w of curcuminoids;
The formula is neutral in taste, i.e. it does not have a sour or bitter-stinging taste, i.e. it is devoid of the characteristic taste of turmeric;
moreover, preferably:
The formulation may contain excipients in the form of stabilizers facilitating emulsification, such as lecithin derivatives, in an amount not exceeding 20% w/w;
The formulation enables the creation of a product containing in a standard portion of 150 g, e.g. a dairy product, preferably fermented, preferably a yogurt, with a daily dose of nutraceutical in the form of curcuminoids within the limits recommended by EFSA, of approximately 3 mg/kg body weight;
The formulation ensures functionality in the range of formulation concentrations not exceeding 1% w/w in relation to the weight of the final product;
The formulation comprises additional ingredients in the form of stabilizers, preferably in the form of sugar polymers, including starch derivatives approved for use in food production, preferably with GRAS status, in the amount not greater than 80% w/w of the above-mentioned formulation.

The inventors unexpectedly found that the yogurt obtained according to the described production method using the technological addition of the formulation of curcuminoids achieves extended shelf life (extended durability), but at the same time it is characterized by a non-obvious and previously undescribed set of functional, organoleptic and technological features, which allows it to be recognized as a new category of dairy products in the form of probiotic-nutraceutical yogurts with extended shelf life, where the individual quality features are defined as:
yogurts-dairy products obtained as a result of natural fermentation of ruminant milk by yogurt bacteria;
probiotic yogurts-yogurt products containing live cultures of probiotic strains in functional quantities (min. 10⁶ CFU/g i.e. 6 log CFU/g, CFU-means colony-forming units);
probiotic-nutraceutical yogurts-yogurt products containing live cultures of probiotic strains in functional amounts (min. 10⁶ CFU/g), containing a nutraceutical substance in a concentration and dose that ensures, with an average daily intake of the indicated portion, a functional effect resulting from probiotic and nutraceutical functionality;
extended shelf life—the period of maintaining the desired organoleptic, sensory and microbiological features characteristic of the product obtained without any additive that extends the shelf life—in this case, a nutraceutical being a technological additive in the form of a formulation of curcuminoids, longer for a product obtained with the addition of the nutraceutical than for the product without the addition of the nutraceutical.

Unexpectedly, such a technological additive ensures an extended shelf life, extends the stability and prolongated durability of the product and enables longer storage, and therefore replaces preservatives, while being a fully natural product that provides nutraceutical properties to fermented milk products containing it, resulting in improved probiotic-nutraceutical fermented milk products with extended shelf life and an improved taste.

### DESCRIPTION OF DRAWINGS

For a better understanding of the invention, it has been illustrated in the nonlimiting embodiments and in the accompanying figures, in which:

FIG. 1 presents the growth dynamics of probiotic cultures over time: a—YC-X16, b—Lactobacillus acidophilus, c-Lactobacillus casei, d-Lactobacillus johnsonii, e-Bifidobacterium animalis subsp. lactis BB-12 (hereinafter referred to as Bifidobacterium BB-12).

FIG. 2 presents the sensory evaluation of yogurt a) after 7, b) after 14, c) after 21, d) after 28 days of storage from production, C-control without curcuminoids, N-addition of the dispersible formulation of curcuminoids (N), TE-addition of curcuminoid extract.

### EXAMPLES

The following examples were presented merely to illustrate the invention and to clarify its various aspects, but are not intended to be limitative, and should not be equated with all its scope, which is defined in the appended claims. In the following examples, unless it was otherwise indicated, standard materials and methods used in the field were used, or the manufacturers' instructions for specific materials and methods were followed.

### Example 1. Preparation of a Probiotic Yogurt Using the Formulation of Curcuminoids

In order to obtain a probiotic yogurt containing the nutraceutical in the form of curcumin, preparations containing curcumin were analyzed to determine the possibility of using them in the yogurt fermentation process.

A total of 9 substances were analyzed, including commercially available formulations of curcuminoids such as: NovaSOL® (Frutarom, Izrael), Cavacurmin® (Wacker Chemie AG, Germany), TheracurminCP (Theravalues Corp., Japan), Curcugen (DolCas Biotech, USA), C3 (Complex®+Bioperine®, Sabinsa, USA), CurQfen® (Akay-Group, India), NomiCU® L-100 (NomiBiotech Corporation, Poland), the NomiCU® beta formulation according to the PL241417 patent with a content of 20% of curcuminoids and a turmeric rhizome extract standardized to contain 95% curcuminoids.

The process of preparing yogurt requires even mixing of ingredients-therefore, the key feature of technological and functional additives is the solubility in water and/or milk or the ability to create stable dispersions in water and/or milk of the additive added in an amount not exceeding 2% w/w of the additive relative to the final product, more preferably up to about 1% w/w.

Preparations characterized by a low solubility or the lack of the ability to create stable dispersions make the technological process impossible due to the formation of a sediment.

Due to their physical characteristics—i.e. the lack of the ability to create stable dispersions, which constitutes an obstacle to use of the formulation in technological processes related to the production of yogurt, which also negatively affects the organoleptic parameters and makes it difficult to uniformly dose the nutraceutical substance in the process of portioning of the final product, NovaSOL®, Cavacurmin, Curcugen, C3, Curcufen and standardized curcuminoid extract were eliminated. In the case of NovaSOL®, an additional limitation was the relatively low content of curcuminoids, forcing the use of an additive in an amount exceeding 1% w/w of the final product. In the case of all the above-mentioned, an additional aspect limiting the use were the negative sensory features that significantly influenced the organoleptic and sensory parameters of the products, such as the intensive taste and smell characteristic of the curcuminoid extract. A comparison of the quality features of the individual 9 preparations with an indication of discriminating features and desirable features is presented in Table 1 below.

TABLE 1
Presents the comparison of the quality features of the analyzed formulations with an indication
of the discriminating features (italics) and the desired features (underlined and bolded).

| | | | | | | Theracurmin | NOMICU ® | NomiCU ® | extract |
|---|---|---|---|---|---|---|---|---|---|
| | NovaSOL | Cavacurmin | Curcugen | C3 | Curcufen | CP | L-100 | beta | 95% |
| dispersion in water | <50% | <30% | <20% | <10% | <20% | 100% | 100% | 100% | <10% |
| dispersion stability | NO | NO | NO | NO | NO | YES | YES | YES | NO |
| curcuminoid content | 6% | 15% | 50% | 95% | 39% | 33% | 28.6% | 20% | 95% |
| neutrality of taste | NO | NO | NO | NO | NO | YES | YES | YES | NO |
| content of essential oils | <0.1% | <0.01% | >0.1% | >0.1% | >0.1% | <0.01% | <0.01% | <0.01% | >0.1% |

However, all nine formulations of curcuminoids were tested in the yogurt-making pilot experiment.

Milk with 3.2% fat content was mixed with skimmed milk powder, pasteurized at 85° C. for 30 min and then cooled to 40° C.

The milk was then inoculated with a single YC-X16 starter culture (Chr. Hansen, Hvidovre, Denmark), Lactobacillus delbrueckii, Streptococcus thermophilus and Bifidobacterium BB-12 (ATCC) to produce starter cultures in the form of a mass activated at 40° C. for 10 h.

The specific curcuminoid preparation was added to the chilled milk prepared as above at final concentrations corresponding to 0%, 0.10%, 0.15%, 0.20% and 0.25% w/w of curcuminoids.

Each batch of milk was inoculated with a previously prepared activated starter culture, in all possible combinations (formulation-starter culture), mixed and fermented at 40° C. for 10 hours. The final products prepared were mixed and poured into 100 ml plastic cups and cooled to 5° C.

After fermentation, the samples were subjected to organoleptic analysis (smell, taste) and features such as uniformity of color and precipitation/presence of sediment were assessed. A trained panel of sensory experts participated in the assessment.

The presence/precipitation of precipitate was considered a disqualification criterion for a given preparation, as this phenomenon makes it impossible to obtain a uniform concentration of the nutraceutical active substance, and additionally constitutes a technological problem due to the fact that precipitate may formed in the technological lines (pumps, dispensers), disturbing the flow of a liquid.

The experiment carried out showed that regardless of the starter strain used, the possibility of using it in the production of fermented dairy products is limited only to preparations characterized by a high level of dispersion. An additional factor limiting the potential use is the unfavorable effect on sensory and organoleptic parameters resulting from the presence of too high (greater than <0.01%) aromatic compounds, typical of the starting raw material for obtaining curcuminoid extract.

In subsequent experiments, due to the high content of curcuminoids and positive results of sensory and organoleptic parameters, the following formulations were used: NomiCU® L-100 (hereinafter referred to as N), NomiCU® beta formulation (hereinafter referred to as N1) and TheracurminCP (hereinafter referred to as N2), as well as a standardized extract with 95% curcuminoids (hereinafter referred to as TE) as a negative control.

### Example 2. Comparative Analysis of Formulations of Dispersible Curcuminoids

In order to determine the necessary features of a technological additive in the form of a formulation of curcuminoids, important from the point of view of the possibility of use in fermented dairy products, a comparative analysis of formulations of curcuminoids was performed (Table 2).

TABLE 2
Comparative analysis of formulations of curcuminoids.

| | Theracurmin CP | NOMICU ® L-100 | NomiCU ® beta | extract 95% |
|---|---|---|---|---|
| | (N2) | (N) | (N1) | (TE) |
| dispersion in full-fat | 100% | 100% | 100% | <10% |
| cow's milk | | | | |
| sedimentation in | 17.3% | 15.9% | 19.5% | 97.3% |
| accelerated | | | | |
| sedimentation assay 425 | | | | |
| rcf/10 min (% w/w) | | | | |
| curcuminoid content | 33% | 28.6% | 20% | 95% |
| (% w/w) | | | | |
| dispersion particle size | 1-2 μm | 1-2 μm | 1-2 μm | >2 μm |
| range for >95% of | | | | |
| particles | | | | |
| Carrier - main excipient - | maltose 30% | maltodextrin 65% | carboxymethyl- | not present |
| (% w/w) | starch octenyl | | cellulose 44% | |
| | succinate 20.4% | | starch octenyl | |
| | maltodextrin 13.2% | | succinate 20% | |
| | | | hydroxypropyl- | |
| | | | methylcellulose 9% | |
| additional excipients | citric acid 0.4% | saccharoglycerides | beta glucan 4% | not present |
| (% w/w) | | 2% | | |
| | | lecithin 8% | | |
| essential oils content | <0.01% | <0.01% | <0.01% | >0.1% |
| neutrality of taste | YES | YES | YES | NO |

All three, further successfully used dispersible formulations of curcuminoids contain >20% curcuminoids, are characterized by complete dispersion in water and milk, maintaining high stability in conditions of accelerated sedimentation, <20% sedimentation, and the particle size is in the range of 1-2 micrometers.

Main excipients—carriers consist of combinations of sugars and polysaccharides approved for use in food products, including saccharide polymers derived from starch and cellulose. The excipients are preferably selected from maltose, starch derivatives (starch octenylsuccinate, starch octenylsuccinate salt, monostarch phosphate, acetylated starch, acetylated distarch adipate), maltodextrins, cellulose derivatives (carboxymethylcellulose, hydroxypropylmethylcellulose and mixtures thereof.

Additional excipients in the form approved for use in food products act as stabilizers and constitute less than 20% w/w, more preferably no more than 10% w/w of the formulation. Additional excipients are preferably selected from citric acid, saccharoglycerides, beta glucan, lecithin and mixtures thereof.

All three dispersible formulations of curcuminoids are made in a process that allows for high purification of curcuminoids in the extraction process and are obtained from curcuminoid extract with a content of min. 95% curcuminoids. According to the characteristics, the process does not cause chemical changes in the structure of curcuminoids and is not an encapsulation process (curcuminoid molecules are not enclosed in a coat of stabilizing substances, but aggregated). The process of obtaining all three formulations involves the same 4 basic steps:
(1) extraction of curcuminoids from dried rhizome using an organic solvent;
(2) dissolving the standardized extract in an organic solvent;
(3) homogenization of the curcuminoid solution with a hydrated sugar (saccharide) bed;
(4) drying the homogenate to obtain a fine powder, preferably by spray drying.

The extraction process and subsequent drying allows the elimination of essential oils contained in curcuminoid extracts, which reduces the unpleasant odor and bitter taste characteristic of curcuminoid extract to a level undetectable by a panel of sensory experts.

In the light of the above data, dispersible formulations of curcuminoids, which can be used as a technological additive of dispersible curcuminoids for the purpose of prolonging durability of dairy products, especially fermented dairy products, while advantageously obtaining probiotic-nutraceutical products, meet the following characteristics:
curcuminoid content >20% w/w;
carrier—the main substance stabilizing curcuminoids is in the form of sugars/polysaccharides or their mixture in the range of 60-70% w/w;
non-sugar stabilizing substances are <10% w/w;
the formulation shows complete dispersion in water and solutions in which the dissolving phase is water;
sedimentation of the formulation in the accelerated sedimentation assay 425 rcf 10 min is <20%.

An additional feature, desirable from the point of view of universal use in food products, is the neutral taste, confirmed by a panel of sensory experts, that does not affect the taste parameters of the target dairy product.

### Example 3. Comparison of the Stability of Pre- and Post-Fermentation Addition of Formulations of Curcuminoids

In order to determine whether the stability of curcuminoids in dairy products is related to the lactic fermentation process by probiotic bacteria or is independent of this process, an experiment was performed comparing the addition of curcuminoids at the pre-(variant A) and post-(variant B) fermentation stage.

Milk with 3.2% fat content was mixed with skimmed milk powder, pasteurized at 85° C. for 30 min and then cooled to 40° C.

The milk was then inoculated with a single YC-X16 starter culture (Chr. Hansen, Hvidovre, Denmark), Lactobacillus delbrueckii, Streptococcus thermophilus and Bifidobacterium BB-12 (ATTC) to produce a starter culture in the form of a mass activated at 40° C. for 10 h.

Then the samples were divided into variant A and variant B.

In variant A, a specific curcuminoid preparation was added to the chilled milk prepared as above at a final concentration corresponding to 0.15%, 0.20% and 0.25% w/w of curcuminoids in all possible combinations (formulation-starter culture), mixed and fermented at the temperature of 40° C. for 10 hours. The final products prepared were mixed and poured into 100 ml plastic cups and cooled to 5° C.

In variant B, the cooled milk was mixed and fermented at 40° C. for 10 hours. Subsequently, after cooling to 5° C., a specific curcuminoid preparation was added at a final concentration corresponding to 0.15%, 0.20% and 0.25% w/w of curcuminoids in all possible combinations (formulation-starter culture). The final products prepared were mixed and poured into 100 ml plastic cups and cooled to 5° C.

Samples obtained in variants A and B were stored for 48 hours. At time points 12 h, 24 h and 48 h, analogous samples of variants A and B obtained using the same strain and concentration of curcuminoids were compared in terms of sedimentation and color. No differences were found for any of the samples, what clearly indicates that the lactic fermentation process does not have a significant impact on the stability of curcuminoid dispersion in the fermented dairy products.

Due to the fact that in the technological process, at the post-fermentation stage to probiotic yogurt-like dairy products flavor additives (fruit inserts), aromatic additives, dye and probiotic additives are added, it is reasonable to assume that adding formulations of curcuminoids at this step is compatible with the technological process.

### Example 4. Preparation of a Probiotic Yogurt with a Stable Count of Probiotic Bacteria

Probiotic yogurt containing strains of probiotic bacteria was prepared according to the method described in Example 3 (Variant B) using probiotic cultures a—YC-X16, b—Lactobacillus acidophilus, c—Lactobacillus casei, d—Lactobacillus johnsonii, e—Bifidobacterium BB-12, with the addition of a post-fermentation formulation of curcuminoids.

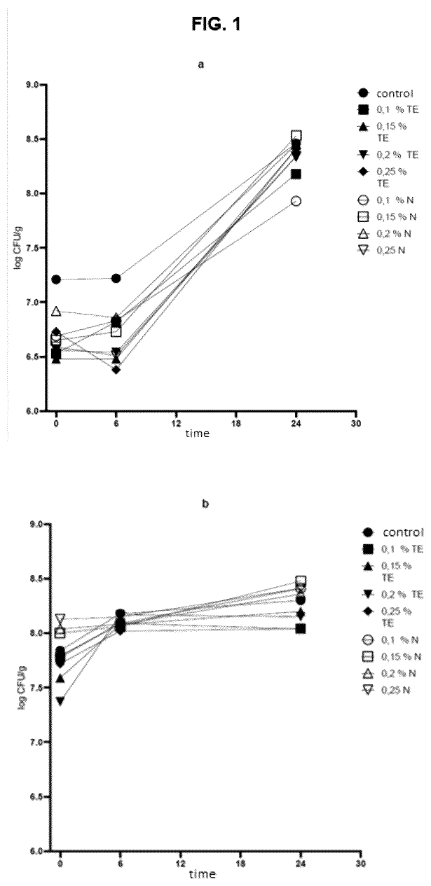

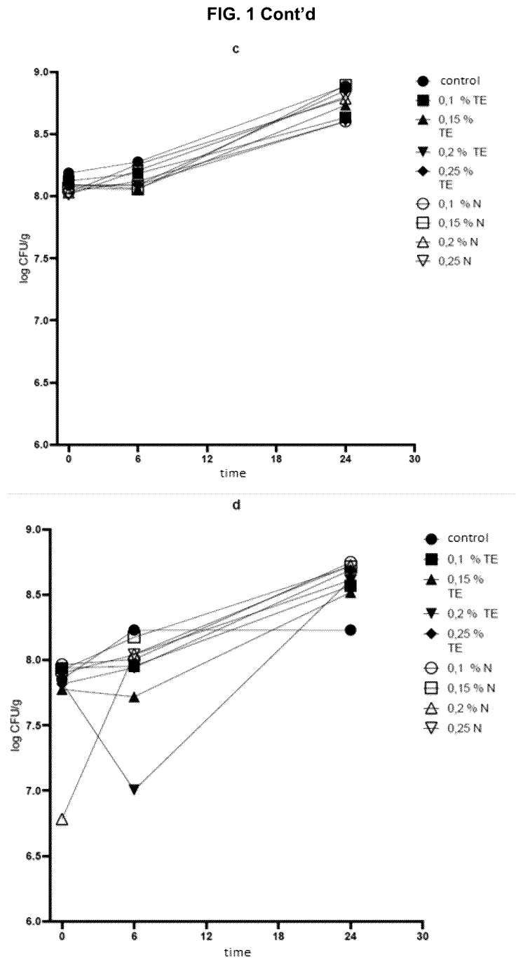

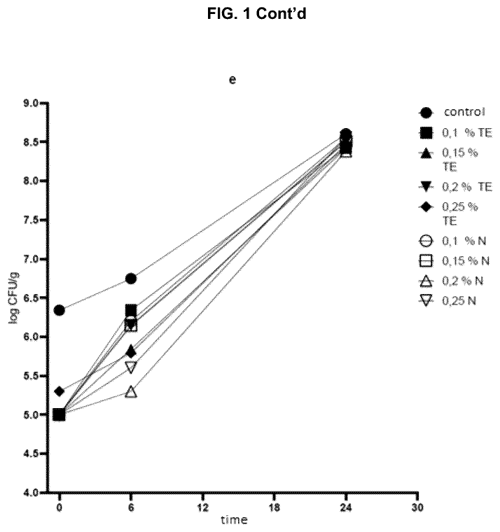

The results of the growth dynamics of probiotic cultures are presented in FIG. 1 a-e. The growth dynamics of probiotic microorganisms within 24 hours indicates that N (technological addition of dispersible curcuminoids) more actively stimulates the development of YC-X16, Lactobacillus acidophilus, Lactobacillus johnsonii and Lactobacillus casei. Simultaneously, in the case of Bifidobacterium BB-12, the same effect is observed for N NOMICUR L-100) and TE (standardized curcuminoid extract). While for the YC-X16 probiotic the active effect of N (0.15-0.25% w/w of curcuminoids) is observed from the first hours, for TE (0.15-0.25% curcuminoids) the active growth of this strain begins after 6 hours and achieves a slightly lower number of microorganisms than for N (log CFU/g 8.34-5.53). In the case of Lactobacillus acidophilus, the growth of bacteria in the case of N (0.15-0.25% w/w of curcuminoids) is dynamic and predictable, while in the case of TE (0.1-0.25% w/w of curcuminoids) there is a rapid increase in the number of viable probiotic cells within the first six hours, leading to a plateau where the number of probiotic bacteria does not change significantly over the next 18 hours. The growth dynamics of Lactobacillus casei microorganisms is slightly different, as FIG. 1 shows that the dose of TE at a concentration of 0.1% w/w of curcuminoids is completely sufficient to obtain an appropriately high result (log CFU/g 8.85), which is lower only than the sample with N (log CFU/g 8.89) for doses of 0.25% w/w of curcuminoids. Increasing the dose of N from 0.1 to 0.25% w/w of curcuminoids reduces the growth activity of the microorganisms Lactobacillus johnsonii, and for TE, on the contrary, increases it slightly. Nevertheless, the total number of probiotic microorganisms is the highest for N (log CFU/g 8.75) at doses of 0.1% w/w of curcuminoids. The effect of TE and N on Bifidobacterium BB-12 is characterized by stable growth dynamics. TE (0.1% w/w of curcuminoids) has the most active effect on probiotic microorganisms within 18-20 hours, which then decreases slightly and is replaced by N (0.1-0.15% w/w of curcuminoids).

The above growth studies of the aforementioned probiotic cultures were performed for N1 (NomiCU® beta) and N2 (TheracurminCP) using the technological addition of dispersible curcuminoids in an amount corresponding to 0.15%, 0.20% and 0.25% w/w of curcuminoids and similar results were obtained as for N.

### Example 5. Antimicrobial Effect of the Addition of Curcuminoids for Inhibiting the Development of Pathogenic Microorganisms

The experiment was conducted to demonstrate the antimicrobial activity of the curcuminoid preparation in the context of the commonly encountered microorganisms responsible for infections of fermented dairy products. Antimicrobial activity of N, N1, N2 and TE was assessed by well diffusion method on Mueller-Hinton agar for bacteria and Sabouraud dextrose agar for yeasts and fungi.

The analysis of the antimicrobial activity was performed using gram-positive (G+) and gram-negative (G−) bacteria, yeasts and fungi on solid media (the results are presented in Table 2′). It was found that dispersible curcuminide preparations show varied antimicrobial activity against a number of microorganisms, which positively affects the microbiological stabilization of nutraceutical-probiotic fermented dairy products, and thus contributes to extending the shelf life of such products in storage conditions. The preparations showed high antimicrobial activity against both G+ bacteria, G− bacteria, yeasts and fungi. The MIC test for the technological addition of dispersible curcuminoids showed the lowest value of MIC 50 (0.09 mg/ml) for C. tropicalis and G+ bacteria (S. pneumoniae), and the highest for MIC 50 (3.6 mg/ml) and MIC 90 (4.2 mg/ml) for G+ bacteria (S. aureus).

TABLE 2′
Antimicrobial activity of NOMICU ® L-100 (N)

| | | | |
|---|---|---|---|
| | Inhibition | MIC 50, | MIC 90, |
| Microorganism | zone mm | mg/mL | mg/mL |

G+ Bacteria

| | | | |
|---|---|---|---|
| Staphylococcus aureus | 6.00 ± 0.03 | 3.6 | 4.2 |
| Listeria monnocytogenes | 7.00 ± 0.02 | 1.8 | 2.5 |
| Streptococcus pneumoniae | 8.00 ± 0.02 | 0.6 | 0.09 |

G− Bacteria

| | | | |
|---|---|---|---|
| Escherichia coli | 0.00 ± 0.00 | 1.8 | 2.5 |
| Pseudomonas aeruginosa | 3.00 ± 0.02 | 0.9 | 1.1 |
| Yersinia enterocolitica | 5.00 ± 0.02 | 0.9 | 1.1 |

Yeasts

| | | | |
|---|---|---|---|
| Candida albicans | 4.00 ± 0.02 | 0.46 | 0.5 |
| Candida glabrata | 5.00 ± 0.02 | 1.8 | 2.5 |
| Candida krusei | 5.00 ± 0.04 | — | — |
| Candida tropicalis | 6.00 ± 0.01 | 0.06 | 0.09 |

Fungi

| | | | | | |
|---|---|---|---|---|---|
| Penicillium chrysogenum | 8.00 ± 0.02 | — | — | — | — |
| Penicillium brevicompactum | 6.00 ± 0.02 | — | — | — | — |
| Penicillium italicum | 7.00 ± 0.02 | — | — | — | — |
| Penicillium aurantiogriseum | 6.00 ± 0.04 | — | — | — | — |

The above antimicrobial activity tests were also performed for N1 (NomiCU® beta) and N2 (TheracurminCP), similar results as for N were obtained.

### Example 6. Production of a Probiotic Yogurt with an Extended Shelf Life and Microbiological Stability

Yogurts were obtained according to the technology described in Example 1 using the technological addition of dispersible curcuminoids (N, N1, N2) and were subjected to microbiological analysis under storage conditions, comparing the samples with the yogurt obtained using the curcuminoid extract (TE) and a control sample without the addition of curcumin. The results of microbiological analysis (plate tests on solid media) of yogurt samples during 28 days of storage are presented in Table 3.

TABLE 3
Number of microorganisms - colony forming units (CFU)
per gram in yogurt samples (ND-not detected).

| | |
|---|---|
| Storage | Value |

| | time | Coliform | BB12 | Yeasts | Fungi |
|---|---|---|---|---|---|
| Sample | (days) | bacteria | count | count | count |
| control | 1 | ND | >7.7 | >7.7 | 6.04 |
| | 7 | ND | >7.7 | >7.7 | 6.2 |
| | 14 | ND | >7.7 | >7.7 | 6.41 |
| | 21 | ND | >7.7 | >7.7 | 6.78 |
| | 28 | ND | >7.7 | >7.7 | >7.7 |
| 0.10% N | 1 | ND | >7.7 | 5.44 | 5.3 |
| | 7 | ND | >7.7 | 5.54 | 5.54 |
| | 14 | ND | >7.7 | 5.78 | 5.85 |
| | 21 | ND | >7.7 | 7.01 | 5.85 |
| | 28 | ND | >7.7 | >7.7 | >7.7 |
| 0.15% N | 1 | ND | >7.7 | 5.3 | 5 |
| | 7 | ND | >7.7 | 5.42 | 5.3 |
| | 14 | ND | >7.7 | 5.54 | 5.3 |
| | 21 | ND | >7.7 | 6.98 | 5.7 |
| | 28 | ND | >7.7 | 7.69 | 6.3 |
| 0.20% N | 1 | ND | >7.7 | 5 | ND |
| | 7 | ND | >7.7 | 5.34 | ND |
| | 14 | ND | >7.7 | 5.54 | 5.21 |
| | 21 | ND | >7.7 | 6.92 | 5.69 |
| | 28 | ND | >7.7 | 7.68 | 6.23 |
| 0.25% N | 1 | ND | >7.7 | 5 | ND |
| | 7 | ND | >7.7 | 5.2 | ND |
| | 14 | ND | >7.7 | 5.51 | 5.21 |
| | 21 | ND | >7.7 | 6.2 | 5.4 |
| | 28 | ND | 6.69 | 6.92 | 6.2 |
| 0.10% TE | 1 | ND | >7.7 | 7.23 | 5.6 |
| | 7 | ND | >7.7 | 7.7 | 5.69 |
| | 14 | ND | >7.7 | >7.7 | 6.26 |
| | 21 | ND | >7.7 | >7.7 | 6.77 |
| | 28 | ND | 5.3 | >7.7 | >7.7 |
| 0.15% TE | 1 | ND | >7.7 | 6.26 | 5.48 |
| | 7 | ND | >7.7 | 6.7 | 5.78 |
| | 14 | ND | >7.7 | 6.86 | 6.3 |
| | 21 | ND | >7.7 | 7.11 | 6.81 |
| | 28 | ND | 5 | >7.7 | 7.01 |
| 0.20% TE | 1 | ND | >7.7 | 5.48 | 5.3 |
| | 7 | ND | >7.7 | 5.54 | 5.78 |
| | 14 | ND | >7.7 | 6.86 | 6.23 |
| | 21 | ND | >7.7 | 7.01 | 6.64 |
| | 28 | ND | 4.7 | 7.68 | 6.8 |
| 0.25% TE | 1 | ND | >7.7 | 5.18 | ND |
| | 7 | ND | >7.7 | 5.48 | ND |
| | 14 | ND | >7.7 | 6.7 | 5.3 |
| | 21 | ND | >7.7 | 6.86 | 5.9 |
| | 28 | ND | 4.5 | 7.11 | 6.69 |

It can be concluded from the obtained results, that the number of live bacteria Bifidobacterium animalis subsp. lactis BB-12 during the first three weeks of storage for all samples was within the limits recommended for functional fermented milk products (7-9 log CFU/g) (Bezerra, Araujo, Santos and Correia, 2015). On day 28 for N at 0.25% w/w of curcuminoids, their number dropped to 6.69 log CFU/g, which, however, still exceeds the minimum allowable level for fermented milk products (6 log CFU/g) (Abadía-García, Cardador, del Campo, Arvízu, Castaño-Tostado, Regalado-Gonzalez and Amaya-Llano, 2013). At the same time, for TE, for all tested concentrations of 0.1-0.25% w/w of curcuminoids, the number of probiotic bacteria was lower than recommended (6 log CFU/g) at the end of the storage period, what may be associated with a higher accumulation of lactic acid (Tirloni, Bernardi, Colombo & Stella, 2015) than when using a dispersible formulation of curcuminoids.

Representatives of coli-form bacteria were not detected in yogurt samples throughout the entire storage period, which is a consequence of the formation of a weakly acidic environment, which adversely affects the emergence and development of this representative of pathogenic microflora. Enumeration of yeasts and microfungi indicates that dispersible formulations of curcuminoids have a stronger effect on them than the curcuminoid extract.

Unlike the technological addition of dispersible curcuminoids, TE curcuminoid extract only slightly inhibits the growth of microorganisms. The curcumin preparation in the form of a technological additive of dispersible curcuminoids has a synergistic effect with the probiotic culture of Bifidobacterium animalis subsp. lactis BB-12, which is a consequence of not only the antimicrobial effect on pathogenic bacterial strains, but also survival of the yogurt starter strain (Khan, Korber, Low, Nicker-son, 2013). What is completely surprising, although the preparation from the curcuminoid extract TE also has antimicrobial effects on yeasts, microfungi and coliform bacteria, it adversely affects the development of a probiotic culture, what significantly shortens the possible shelf life of the product produced using it, as well as reduces the probiotic properties of such product.

The above microbiological analysis tests were also performed for N1 (NomiCU® beta) and N2 (TheracurminCP), with similar results obtained as for N.

### Example 7. Preparation of a Probiotic Yogurt with Favorable Organoleptic (Sensorial) Parameters

Yogurts obtained according to the technology described in Example 1 and yogurts obtained according to Example 1, with proviso that the formulation of curcuminoids was added not before the fermentation process (pre-fermentation), but after its completion (post-fermentation) using a dispersible formulation of curcuminoids (N, N1, N2), have been subjected to organoleptic analysis under storage conditions, comparing the samples with the yogurt obtained using the curcuminoid extract (TE) and a control sample without the addition of curcumin (C).

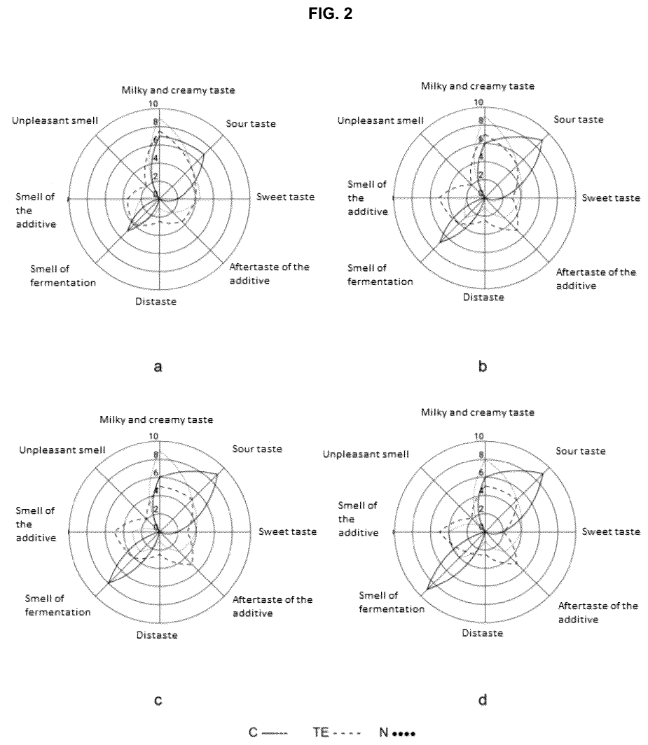

Similar results were obtained for samples with the addition of pre-fermentation and post-fermentation formulations of curcuminoids. Due to the higher compatibility of the procedure related to the use of the post-fermentation additive with the standard procedures used in the industrial production of yogurts, the detailed analysis of the results presented in FIG. 2 concern the post-fermentation additive.

Sensorial evaluation of experimental yogurt samples was performed by a sensory expert panel on days 7, 14, 21 and 28 of storage. The obtained data show that the most pronounced sour milk taste was observed for the control sample (with TE), while the addition of both types of curcumin preparations, curcuminoid extract and the technological addition of dispersible curcuminoids slightly reduced its intensity: especially for the technological additive of dispersible curcuminoids during the first three weeks, but at the end of the storage period the sour taste perception was similar for the technological additive of dispersible curcuminoids and curcuminoid extract, which correlates with the data on the amount of lactic acid formed in the experimental samples. The aroma of sour milk in the case of using the curcuminoid extract was higher for the first three weeks than for the aroma when the technological addition of dispersible curcuminoids was used, but at the end of the storage period it was barely noticeable. Therefore, in the case of the curcuminoid extract, the aroma of this additive was observed already on the 7th day, which intensified during 28 days of storage, what is not a characteristic feature of yogurt (Thomas & Beeren, 2016) and made the yogurt taste unattractive. At the same time, in the case of the technological addition of dispersible curcuminoids, the smell of the additive was discreet after a week of storage and began to intensify slightly only after the second week of storage of the yogurt samples. It should be noted that the use of curcuminoid extract (TE) significantly negatively affected the taste of the yogurt, which began to deteriorate after the first week of storage due to an external bitter taste perception. In the case of the technological addition of dispersible curcuminoids, the aftertaste of the additive was barely noticeable and did not affect the overall taste perception even at the end of the tested storage period (28 days). What was particularly surprising and unexpected, the technological additions of dispersible curcuminoids (N, N1, N2) gave the yogurt a sweet taste and a rich creamy taste that remained stable until the end of storage, while in the case of the curcuminoid extract its intensity dropped to the control level or below. It should be noted that the unexpected feature of yogurts was obtained by using technological additives in the form of formulations of dispersible curcuminoids in the method of their production, i.e. giving them a sweet taste and effectively imitating the taste of milk fat. In particular, obtaining the taste of milk fat is extremely attractive to consumers, and in this case the effect was achieved without adding it. Similarly, when it comes to sweet taste-products with a sweet taste are attractive to the consumer, especially when the sweet taste does not result from the addition of high-calorie sweeteners.

On day 28, an unusual aftertaste was detected for the sample with curcuminoid extract, which may be related to the formation of an insoluble precipitate. In the case of using technological additives of dispersible formulations of curcuminoids, the aftertaste was barely noticeable at the end of the storage period (day 28), which is also an unexpected advantage, as with their use practically no sediment is formed in yogurts. Therefore, according to the results of sensory evaluations, the best was the sample using technological additives of dispersible formulations of curcuminoids with 0.20% w/w curcuminoids, the organoleptic properties of which did not deteriorate even during 28 days of storage. When using the curcuminoid extract as an additive, the taste deteriorated already on the 7th day of storage, what significantly limits the storage period (shelf life) of such a product and does not allow to achieve the intended technological effect. Moreover, when curcuminoid extract was used, yogurt syneresis increased, what was observed on the 28th day of storage.

As a result of the performed experiments, it was found that formulation/s of dispersible curcuminoids having physico-chemical properties and produced with the method containing specific steps as indicated in Example 2 can be use as technological additive/s of dispersible curcuminoids formulation/s for the production of dairy products, wherein the addition provides:
extended shelf life (durability)—i.e. formulation/s of dispersible curcuminoids can be use as the natural preservative, the agent that extends the shelf life of dairy products, especially yogurts;
show antimicrobial properties by inhibiting the development of pathogenic microorganisms;
stimulate the growth of probiotic microorganisms in fermented dairy products, enhancing development of the same;
show a synergistic effect with the probiotic flora of fermented dairy products, strengthening development of the same;
act as the agent improving the organoleptic properties of dairy products, particularly yogurts;
act as the agent that mimics the taste of milk fat in dairy products, particularly yogurts;
act as the agent that increases the sweet taste in dairy products, particularly yogurts;
do not cause bitter or burning taste to dairy products, particularly yogurts;
by the health-promoting properties of curcuminoids and their increased biological availability of dispersible forms, dairy products with nutraceutical properties are obtained, and when used for fermented dairy products-products with probiotic-nutraceutical properties are obtained, particularly probiotic-nutraceutical yogurts.

### LITERATURE

Amalraj, A., Pius, A. Gopi, S. Gopi, S. Biological activities of curcuminoids, other biomolecules from turmeric and their derivatives-A review. Journal of Traditional and Complementary Medicine 2017, 7, 205-233.
Antony, B. Merina, B. Iyer, V. S. Judy, N. Lennertz, K. Joyal, S. A pilot cross-over study to evaluate human oral bioavailability of BCM-95CG (Biocurcumax), a novel bioenhanced preparation of curcumin. Indian J. Pharm. Sci. 2008, 70 (4), 445-449.
Apisariyakul, A.; Vanittanakom, N.; Buddhasukh, D. Antifungal activity of turmeric oil extracted from Curcuma longa (Zingiberaceae). J. Ethnopharmacol. 1995, 49, 163-169.
Batiha G E S, Hussein D E, Algammal A M, George T T, Jeandet P, Al-Snafi A. E, Cruz-Martins N. Application of natural antimicrobials in food preservation: Recent views. Food Control, (2021) 126, 108066.
Dash K K, Fayaz U, Dar A H, Shams R, Manzoor S, Sundarsingh A, Deka P, Khan S A A comprehensive review on heat treatments and related impact on the quality and microbial safety of milk and milk-based products. Food Chemistry Advances (2022) 1, 100041.
Jennings, M. R, Parks R. J. Curcumin as an Antiviral Agent. Viruses. 2020 Oct. 31; 12 (11): 1242.
Kennang Ouamba A J, LaPointe G, Dufour S, Roy D. Optimization of Preservation Methods Allows Deeper Insights into Changes of Raw Milk Microbiota. Microorganisms (2020) 8, 368.
Kukhtyn, M, Horiuk Y, Yaroshenko T, Laiter-Moskaliuk S, Levytska V, Reshetnyk A. Effect of lactic acid microorganisms on the content of nitrates in tomato in the process of pickling. East.-Eur. J. Enterp. Technol., (2018) 1, 69-75.
Madhavi, D. Kagan, D. Bioavailability of a sustained release formulation of curcumin. Integr. Med. (Encinitas) 2014, 13 (3), 24-30.
Mangolim, C. S. Moriwaki, C. Nogueira, A. C. Sato, F. Baesso, L. M. Neto, A. M. Matioli, G. Curcumin-β-cyclodextrin inclusion complex: Stability, solubility, characterisation by FT-IR, FT-Raman, X-ray diffraction and photoacoustic spectroscopy, and food application. Food Chemistry 2014, 153, 361-370.
Marcolino, V. A. Zanin, G. M. Durrant, L. R. Benassi, M. T Matioli, G. Interaction of Curcumin and Bixin with β-Cyclodextrin: Complexation Methods, Stability, and Applications in Food. J. Agric. Food Chem. 2011, 59, 3348-3357.
Mykhalevych A, Polishchuk G, Nassar K, Osmak T, Buniowska-Olejnik M. β-Glucan as a Techno-Functional Ingredient in Dairy and Milk-Based Products-A review. Molecules (2022) 27, 6313.
Petrova P, Ivanov I, Tsigoriyna L, Valcheva N, Vasileva E, Parvanova-Mancheva T, Arsov A, Petrov K. Traditional Bulgarian Dairy Products: Ethnic Foods with Health Benefits. Microorganisms (2021) 9,480.
Purpura, M., Lowery, R. P. Wilson, J. M. Mannan, H. Münch, G. Razmovski-Naumovski, V. Analysis of different innovative formulations of curcumin for improved relative oral bioavailability in human subjects. Eur. J. Nutr. 2018, 57 (3), 929-938
Ruby, A. J.; Kuttan, G.; Baby, K. D.; Rajasekharan, K. N.; Kuttan, R. Anti-tumour and antioxidant activity of natural curcuminoids. Cancer Lett. 1995, 94, 79-83.
Jäger, R. Lowery, R. P. Calvanese, A. V. Joy, J. M. Purpura, M. Wilson, J. M. Comparative absorption of curcumin formulations. Nutrition Journal 2014, 13:11, 1-8.
Sasaki, H. Sunagawa, Y. Takahashi, K. Imaizumi, A. Fukuda, H. Hashimoto, T. Innovative preparation of curcumin for improved oral bioavailability. Biol. Pharm. Bull. 2011, 34 (5), 660-665.
Schiborr, C. Kocher, A. Behnam, D. Jandasek, J. Toelstede, S. Frank, J. The oral bioavailability of curcumin from micronized powder and liquid micelles is significantly increased in healthy humans and differs between sexes. Mol. Nutr. Food Res. 2014, 58 (3), 516-527.
Shoba, G. Joy, D. Joseph, T. Majeed, M. Rajendran, R. Srinivas, P. S. Influence of piperine on the pharmacokinetics of curcumin in animals and human volunteers. Planta. Med. 1998, 64 (4), 353-356.
Var I, Şahan N, Kabak B, Golge O. The effects of natamycin on the shelf life of yogurt. Arch.fur Leb., (2004) 55, 7-9.
Wang Y J, Pan M H, Cheng A L, Lin L I, Ho Y S, Hsieh C Y, Lin J K. Stability of curcumin in buffer solutions and characterization of its degradation products. J Pharm Biomed Anal. 1997; 15:1867-1876.
Yamamoto, H.; Hanada, K.; Kawasaki, K.; Nishijima, M. Inhibitory effect on curcumin on mammalian phospholipase D activity. FEBS Lett. 1997, 417, 196-198.
Zapaśnik A, Sokołowska B, Bryła M. Role of Lactic Acid Bacteria in Food Preservation and Safety. Foods (Basel, Switzerland) (2022) 11, 1283.

1. A use of a formulation of curcuminoids as a technological additive extending the shelf life of fermented dairy products,
wherein the formulation of curcuminoids is a composition that:
in dry form comprises at least 20% to 25% w/w of curcuminoids;
comprises no more than 0.01% w/w of essential oils;
comprises a carrier of curcuminoids in the range of 60-70% w/w of the formulation, wherein the carrier of curcuminoids are sugars, polysaccharides, including saccharide polymers derived from starch, cellulose or mixtures thereof, approved for use in food products;
comprises up to 20% w/w of an additional non-sugar excipient in a form approved for use in food products,
the formulation disperses in water in 98% to 100%, wherein in the accelerated sedimentation assay when centrifuged at a relative centrifugal force of 425 RCF for 10 min no less than 80% of the formulation remains in the solution;
the size of the particles of the dispersion for at least 95% of the particles is 1-2 μm; and
the formulation of curcuminoids is neutral in taste.

2. The use according to claim 1, wherein the carrier of curcuminoids is a member selected from the group consisting of maltose, starch derivatives, starch octenylsuccinate, starch octenylsuccinate salt, monostarch phosphate, acetylated starch, acetylated distarch adipate, maltodextrin, cellulose derivatives, carboxymethylcellulose, hydroxypropylmethylcellulose and mixtures thereof.

3. The use according to claim 1, wherein the additional non-sugar excipients are members selected from the group consisting of citric acid, saccharoglycerides, beta glucan, lecithin and mixtures thereof.

4. The use according to claim 1, wherein additional non-sugar excipients are in the amount of up to 10% w/w of the formulation.

5. The use according to claim 1, wherein the formulation of curcuminoids constituting the technological additive, when in dry form, is in the form of a powder, granules or a mixture thereof.

6. The use according to claim 1, wherein the fermented dairy product is a member selected from the group consisting of yogurt, kefir, buttermilk, fermented milk, sour cream, ryazhenka, curdled milk, ajran, kumis, and acidophilous milk.

7. The use according to claim 1, wherein the formulation of curcuminoids as the technological additive extending the shelf life of fermented dairy products is used in an amount of 0.1-0.25% w/w of curcuminoids in the fermented dairy product.

8. The use according to claim 1, wherein the fermented dairy product is a probiotic-nutraceutical product comprising:
live cultures of probiotic strains in an amount of at least 10⁶ CFU/g, and
in a 150 g portion of the fermented dairy product it contains a dose of a nutraceutical in the form of curcuminoids, providing the person consuming it, the amount of 2-4 mg/kg body weight, and the amount of 3±10% mg/kg body weight.

9. The use according to claim 1, wherein the formulation of curcuminoids is the formulation in which the carrier of curcuminoids is a mixture of maltose, starch octenylsuccinate, and maltodextrin, and the additional excipient is citric acid.

10. The use according to claim 1, wherein the formulation of curcuminoids is the formulation in which the carrier of curcuminoids is maltodextrin and the additional excipient is a mixture of saccharoglycerides and lecithin.

11. The use according to claim 1, wherein the formulation of curcuminoids is the formulation in which the carrier of curcuminoids is a mixture of carboxymethylcellulose, starch octenylsuccinate, and hydroxypropylmethylcellulose,
and the additional excipient is beta glucan.

12. The use according to claim 1, wherein the formulation of curcuminoids is obtained in a process, in which:
dried Curcuma longa rhizome is subjected extraction for curcuminoids using an organic solvent, obtaining a highly purified curcuminoid extract with a content of min. 95% curcuminoids, which extract is then dissolved in an organic solvent;
homogenization of the curcuminoids solution with a hydrated bed of the carrier of curcuminoids consisting of sugars, polysaccharides including saccharide polymers derived from starch, cellulose or mixtures thereof, approved for use in food products is carried out, preferably supplementing the solution with additional non-sugar excipients; and
the obtained homogenate solution is dried.

13. A method of extending the shelf life of a fermented dairy product comprising a step, in which:
a) before the fermentation process of the dairy product, or
b) after the fermentation process of the dairy product
adding a water dispersible formulation of curcuminoids in an amount providing 0.1-0.25% w/w of curcuminoids in the fermented dairy product as a technological additive extending the shelf life of fermented dairy products; the amount of the additive is between 1% and 2% w/w of the final dairy product,
wherein the technological additive of the formulation of curcuminoids extending the shelf life of fermented dairy products is the formulation of curcuminoids that:
in dry form comprises at least 20% to 25% w/w of curcuminoids;
comprises no more than 0.01% w/w of essential oils;
comprises a carrier of curcuminoids in the range of 60-70% w/w, wherein the carrier of curcuminoids are sugars, polysaccharides, including saccharide polymers derived from starch, cellulose or mixtures thereof, approved for use in food products;
comprises up to between 10% w/w and 20% w/w of an additional non-sugar excipient in a form approved for use in food products;
disperses in water in at least 98%, preferably shows 100% dispersion, wherein in the accelerated sedimentation assay, during centrifugation at a relative centrifugal force of 425 RCF for 10 min, no less than 80% of the formulation remains in the solution;
the particle size of the dispersion for at least 95% of the particles is 1-2 μm; and
the formulation of curcuminoids is neutral in taste.

14. The method according to claim 13, wherein the carrier of curcuminoids is a member selected from the group consisting of maltose, starch derivatives, starch octenylsuccinate, starch octenylsuccinate salt, monostarch phosphate, acetylated starch, acetylated distarch adipate, maltodextrin, cellulose derivatives, carboxymethylcellulose, hydroxypropylmethylcellulose and mixtures thereof.

15. The method according to claim 13, wherein the additional non-sugar excipients are members selected from the group consisting of citric acid, saccharoglycerides, beta glucan, lecithin and mixtures thereof.

16. The method according to claim 13, wherein the formulation of curcuminoids constituting the technological additive, when is in dry form, is in the form of powder, granules or a mixture thereof.

17. The method according to claim 13, wherein the fermented dairy product is a member selected from the group consisting of yogurt, kefir, buttermilk, sour milk, sour cream, ryazhenka, curdled milk, ajran, kumis, and acidophilous milk.

18. The method according to claim 13, wherein the fermented dairy product is a probiotic-nutraceutical product comprising
live cultures of probiotic strains in an amount of at least 10⁶ CFU/g, and
in a 150 g portion of the fermented dairy product it contains, a dose of a nutraceutical in the form of curcuminoids, providing a person consuming it the amount of 2-4 mg/kg body weight, and the amount of 3±10% mg/kg body weight.

19. A fermented dairy product with extended shelf life prepared by the method as defined in claim 13.

20. The fermented dairy product according to claim 19, wherein the fermented dairy product [it] is a member selected from the group consisting of yogurt, kefir, buttermilk, sour milk, sour cream, ryazhenka, curdled milk, ayran, kumis, and acidophilous milk.

21. The fermented dairy product according to claim 20, wherein the fermented dairy product [it] is a probiotic-nutraceutical product comprising
live cultures of probiotic strains in an amount of at least 10⁶ CFU/g, and in a 150 g portion of the fermented dairy product, it contains a dose of a nutraceutical in the form of curcuminoids, providing a person consuming it the amount of 2-4 mg/kg body weight, preferably the amount of 3±10% mg/kg body weight.

22. A use of formulation of curcuminoids as an agent improving the organoleptic and taste properties of a fermented dairy product,
wherein the formulation of curcuminoids is a composition that:
in dry form comprises at least 20% to 25% w/w of curcuminoids;
comprises no more than 0.01% w/w of essential oils;
comprises a carrier of curcuminoids in the range of 60-70% w/w of the formulation, wherein the carrier of curcuminoids are sugars, polysaccharides, including saccharide polymers derived from starch, cellulose or mixtures thereof, approved for use in food products;
comprises up to 20% w/w of an additional non-sugar excipient in a form approved for use in food products;
the formulation disperses in water in 98% to 100%, wherein in the accelerated sedimentation assay, during centrifugation at a relative centrifugal force of 425 RCF for 10 min, no less than 80% of the formulation remains in the solution;
the size of the particles of the dispersion for at least 95% of the particles is 1-2 μm;
the formulation of curcuminoids is neutral in taste;
wherein the agent improving the organoleptic and taste properties of the fermented dairy product is used as an agent imitating taste of milk fat and/or as an agent increasing the perception of sweet taste.

23. The use according to claim 22, wherein the carrier of curcuminoids is a member selected from the group consisting of maltose, starch derivatives, starch octenylsuccinate, starch octenylsuccinate salt, monostarch phosphate, acetylated starch, acetylated distarch adipate, maltodextrin, cellulose derivatives, carboxymethylcellulose, hydroxypropylmethylcellulose and mixtures thereof.

24. The use according to claim 22, wherein the additional non-sugar excipients are members selected from the group consisting of citric acid, saccharoglycerides, beta glucan, lecithin and mixtures thereof.

25. A use of formulations of curcuminoids as an agent with antimicrobial activity against pathogenic microorganisms and, at the same time, stimulating the growth of probiotic microorganisms in fermented dairy products,
wherein the formulation of curcuminoids is a composition that:
in dry form comprises at least 20% to 25% w/w of curcuminoids;
comprises no more than 0.01% w/w of essential oils;
comprises a carrier of curcuminoids in the range of 60-70% w/w of the formulation, wherein the carrier of curcuminoids are sugars, polysaccharides, including saccharide polymers derived from starch, cellulose or mixtures thereof, approved for use in food products;
comprises up to 20% w/w of an additional non-sugar excipient in a form approved for use in food products;
the formulation disperses in water by 98% to 100%, wherein in the accelerated sedimentation assay, during centrifugation at a relative centrifugal force of 425 RCF for 10 min, no less than 80% of the formulation remains in the solution;
the particle size of the dispersion for at least 95% of the particles is 1-2 μm;
the formulation of curcuminoids is neutral in taste.

26. The use according to claim 25, wherein the carrier of curcuminoids is a member selected from the group consisting of maltose, starch derivatives, starch octenylsuccinate, starch octenylsuccinate salt, monostarch phosphate, acetylated starch, acetylated distarch adipate, maltodextrin, cellulose derivatives, carboxymethylcellulose, hydroxypropylmethylcellulose and mixtures thereof.

27. The use according to claim 25, wherein the additional non-sugar excipients are members selected from the group consisting of citric acid, saccharoglycerides, beta glucan, lecithin and mixtures thereof.

28. The use according to claim 25, wherein the pathogenic microorganisms are selected from one of G+ bacteria, G− bacteria, yeast, or fungi: wherein
G+ bacteria is one member selected from Staphylococcus aureus, Listeria monnocytogenes, Streptococcus pneumoniae;
G− bacteria is one member selected from Escherichia coli, Pseudomonas aeruginosa, Yersinia enterocolitica;
yeasts is one member selected from Candida albicans, Candida glabrata, Candida krusei, Candida tropicalis,
fungi is one member selected from Penicillium chrysogenum, Penicillium brevicompactum, Penicillium italicum, Penicillium aurantiogriseum;
and in that the probiotic microorganisms are selected from Lactobacillus delbrueckii, Streptococcus thermophilus, Bifidobacterium animalis subsp. lactis, Lactobacillus acidophilus, Lactobacillus casei, and Lactobacillus johnsonii.
